SEQUENCE LISTING (22 sequences)

SEQ ID NO: 1 - protein, 121 aa, Macaca mulatta
VQLQESGPGLVKPSETLSLTCAVSGDSISTGYDWTWIRQPPGKGLEWIGYIGGSSGNTNYNPSLKSRVTISKDTSKNQFSLKLSSVTAADTAVYYCARDGTRVYYYGLDSWGQGVVVTVSS

SEQ ID NO: 2 - DNA, 364 bp, Macaca mulatta
gtgcagctgcaggagtcgggcccagggctggtgaagccttcggagaccctgtccctcacctgcgctgtctctggtgactccatcagcactggttatgactggacctggatccgccagcccccagggaaagggctggagtggattgggtatatcggtggtagtagtggtaacaccaactacaatccctccctcaagagtcgagtcaccatttcaaaagacacgtccaagaaccagttctccctgaagctgagttctgtgaccgccgcggacaccgccgtgtattactgtgcgagagatggcacccgggtgtattactacggtttggattcctggggccaaggggtcgtcgtcaccgtctcctcag

SEQ ID NO: 3 - protein, 110 aa, Macaca mulatta
ETVVTQEPSLSVSPGGTVTLTCGLSSGSVSTSNYPSWFQQTPGQAPRMLIYSTNIRPSGVPNRFSGSILGNKAALTITGAQADDESDYYCFFSMVSGTSVFGGGTRLTVL

SEQ ID NO: 4 - DNA, 331 bp, Macaca mulatta
gagactgtggtgacccaggagccatcgttgtcagtgtcccctggaggaacagtcacactcacctgtggcctgagctctggctcagtctctaccagcaactaccccagctggttccagcagaccccaggccaggctccacgcatgctcatctacagtacaaatattcgcccttctggggtccctaatcgcttctctggctccatccttgggaacaaagctgccctcaccatcacgggggctcaggcagacgatgaatctgattactactgtttcttctccatggttagtggcacttcggttttcggcggagggacccggctgaccgtcctag

SEQ ID NO: 5 - protein, 116 aa, Macaca mulatta
EVQLVESGGGLVQPGGSLRLSCAASGFTFSSYDMSWVRQAPGKGLEWVSSISSVTSYISYADSVKGRFTISRDNAKNSLSLQMNSLKTEDTAVYYCTTENWNFDFWGQGVLVTVSS

SEQ ID NO: 6 - DNA, 349 bp, Macaca mulatta
gaggtgcaactggtggagtctgggggaggcttggtccagcctggagggtccctgagactctcctgtgcagcctctggattcaccttcagtagttacgacatgagctgggtccgccaggctccgggaaaggggctggagtgggtctcatccattagtagtgttactagttacatatcctacgctgactccgtgaagggccgattcaccatctccagagacaacgccaagaactcgctgtctctgcaaatgaacagcctgaaaaccgaggacacggccgtatattactgtactactgaaaactggaactttgacttctggggccagggagtcctggtcaccgtctcctcag

SEQ ID NO: 7 - protein, 113 aa, Macaca mulatta
DVVMTQSPLSLATTPGQPASISCRSSQSLVHSNGNTYLSWYQQKPGQPPRLLIYEVSNRDSGVPDRFSGSGAGTDFTLKISRVEAEDVGVYYCGQGTHWPPFTFGPGTKLDIK

SEQ ID NO: 8 - DNA, 340 bp, Macaca mulatta
gatgttgtgatgactcagtctccactctccctggccaccacccctggacagccggcctccatctcctgcaggtctagtcaaagccttgtacacagtaatggaaacacctacttgagttggtatcagcagaagccaggccaacctccaaggctcctgatttatgaggtttctaaccgggactctggggtcccagacagattcagcggcagtggggcagggacagatttcacactgaaaatcagcagagtggaggctgaggatgttggggtttattactgcgggcaaggtacacactggcctccgttcactttcggccccgggaccaaactggatatcaaac

SEQ ID NO: 9 - protein, 121 aa, Macaca mulatta
QVQLQESGPGLVKPSETLSLSCAVSGGSFSDYWWGWIRQSPGKGLEWIGSIYGSSGRTAYSPSLKSRATISRDTSKNQLFLRLTSVTAADTGVYYCTNERFYGDSNRFDVWGPGVLVTVSS

SEQ ID NO: 10 - DNA, 364 bp, Macaca mulatta
caggtgcagctgcaggagtcgggcccaggactggtgaagccttcggagaccctgtccctctcctgcgctgtctctggtggctccttcagcgattactggtggggctggatccgccagtccccagggaaggggctggagtggattgggagtatctatggtagtagtgggcgaaccgcatacagcccctccctcaaaagtcgagccaccatttcaagagacacgtccaagaaccagctcttcctgaggctgacctctgtgaccgccgcggacacgggagtctactactgtaccaacgagaggttctacggtgacagcaaccggttcgatgtctggggcccgggagtcctggtcaccgtctcctcag

SEQ ID NO: 11 - protein, 115 aa, Macaca mulatta
QPVLTQPTSLSASPGASARLTCTLSSGISVGNYRIFWYQQKPGSPPRYLLNYHTDSDKHQGSGVPSRFSGSKDASANAGILLISGLQSEDEADYYCMIWHNNVDVFGGGTKLTVL

SEQ ID NO: 12 - DNA, 346 bp, Macaca mulatta
cagcctgtgctgactcagccaacctccctctcagcatctcctggagcatcagccagactcacctgcaccttgagcagtggcatcagtgttggtaattacaggattttctggtaccagcagaagccagggagtcctccccggtatcttctgaactaccacacagactcagataagcaccagggctctggagtccccagtcgcttctctggatccaaagatgcttcggccaatgcagggattttactcatctctgggctccagtctgaggatgaggctgactattactgtatgatttggcacaacaatgttgatgtgttcggaggtggcaccaagttgaccgtcctcg

SEQ ID NO: 13 - protein, 119 aa, Macaca mulatta
QVQLVQSGAEVKKPGASVKLSCKASGYTFTSFYINWVRQAPGQVLEWMGWINPSNGDTGYAQKFQGRVTMSRDTSTSTAYMELSSLRSEDTAVYYCTRETVGSGTFDCWGQGVLVTVSS

SEQ ID NO: 14 - DNA, 358 bp, Macaca mulatta
caggtccagctggtgcagtccggggctgaggtgaagaagcctggggcctcagtgaaactctcctgcaaggcttctggttacacctttaccagcttctatataaactgggtgagacaggcccctggacaagtgcttgagtggatgggatggattaaccctagcaatggtgatacaggctacgcacagaaattccagggcagagtcaccatgtccagggacacgtcgacgagtacagcctacatggagctgagcagcctgagatctgaggacacggccgtgtattattgtacgagagagactgttggtagtggaacctttgactgctggggccagggagtcctggtcaccgtctcctcag

SEQ ID NO: 15 - protein, 112 aa, Macaca mulatta
DIVMTQTPLSLSVTLGEPASISCRSSQSLVYSAGKTYLDWYLQKPGQSPQLLIYLVSKRASGVPDKFSGSGSGTDFTLKISRVEAEDVGVYYCMQALRSPLTFGGGTKVELK

SEQ ID NO: 16 - DNA, 337 bp, Macaca mulatta
gatattgtgatgacccagactccactctccctgtccgtcacccttggagagccggcctccatctcctgcaggtctagtcagagccttgtatacagtgctggaaaaacctatttggattggtacctgcagaagccaggccagtctccacagctcctaatatatttggtttccaaacgggcctctggagtcccagacaagttcagtggcagtgggtcaggcactgatttcacactgaaaatcagcagggtggaggctgaggatgttggggtttattactgcatgcaagctctacggtctccgctcactttcggcggagggaccaaggtggagctcaaac

SEQ ID NO: 17 - protein, 330 aa, Homo sapiens [sequence omitted]

SEQ ID NO: 18 - DNA, 990 bp, Homo sapiens
gcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 19 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 318 bp, Homo sapiens
actgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 21 - protein, 106 aa, Homo sapiens [sequence omitted]

SEQ ID NO: 22 - DNA, 318 bp, Homo sapiens
cgtcagcccaaggctgccccctcggtcactctgttcccaccctcgagtgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

(19) United States
(12) Patent Application Publication
(10) Pub. No.: US 2024/0158480 A1
(43) Pub. Date: May 16, 2024

(54) ANTI-NIPAH VIRUS MONOCLONAL ANTIBODY HAVING NEUTRALIZATION ACTIVITY AND APPLICATION

(71) Applicant: ACADEMY OF MILITARY MEDICAL SCIENCE, PLA, Beijing (CN)

(72) Inventors: Wei Chen, Beijing (CN); Changming Yu, Beijing (CN); Yujiao Liu, Beijing (CN); Pengfei Fan, Beijing (CN); Guanying Zhang, Beijing (CN); Yaohui Li, Beijing (CN); Jianmin Li, Beijing (CN); Xiangyang Chi, Beijing (CN); Meng Hao, Beijing (CN); Ting Fang, Beijing (CN); Yunzhu Dong, Beijing (CN); Xiaohong Song, Beijing (CN); Yi Chen, Beijing (CN); Shuling Liu, Beijing (CN)

(73) Assignee: ACADEMY OF MILITARY MEDICAL SCIENCE, PLA, Beijing (CN)

(21) Appl. No.: 18/005,978

(22) PCT Filed: Jun. 26, 2021
(86) PCT No.: PCT/CN2021/102585

§ 371 Date: Jan. 19, 2023

(30) Foreign Application Priority Data
Jul. 22, 2020 (CN) 202010711672.9

(51) Int. Cl. C07K 16/10; A61P 31/14

(52) U.S. Cl. CPC C07K 16/1027; A61P 31/14

(57) ABSTRACT

Disclosed in the present invention is an anti-Nipah virus monoclonal antibody having neutralization activity. The antibody consists of a monkey-derived variable region and a human constant region, and both light and heavy chains of the monkey-derived variable region have unique CDR regions. The antibody provided by the present invention has an excellent antigen binding capability, and has good binding activity with Bangladesh Nipah virus and Malaysia Nipah virus glycoprotein G. The antibody can effectively neutralize the Nipahpseudovirus. Moreover, the neutralization activity of the antibody is enhanced as the concentration of the antibody increases, and nearly 100% neutralization of the Nipahpseudovirus can be achieved at a concentration of 1 μg/mL. Also disclosed in the present invention is an application of the monoclonal antibody against the Nipah virus glycoprotein G in preparation of a Nipah virus treatment drug.

### CROSS-REFERENCE TO RELATED APPLICATIONS

This application is the U.S. national phase of International Patent Application No. PCT/CN2021/102585, filed on Jun. 26, 2021, which claims priority to Chinese Patent Application No. 202010711672.9, filed on Jul. 22, 2020, the contents of which are incorporated herein by reference in their entirety.

### REFERENCE TO A SEQUENCE LISTING

This application incorporates by reference the Sequence Listing submitted in Computer Readable Form as file SeqList_176495-00700, created on Jul. 5, 2023 and containing 19,717 bytes.

### TECHNICAL FIELD

The invention discloses a group of monoclonal antibodies, which belongs to the field of immunology and microbiology.

### BACKGROUND TECHNOLOGY

Nipah virus (NiV) is a single negative-stranded RNA virus, belonging to the genus Henipavirus of the family Paramyxoviridae. NiV is a zoonotic virus that can be infected by direct contact and can cause fatal respiratory and neurological disease. The natural hosts of NiV is fruit bats. NiV can be transmitted through fruit bat-pig-human transmission and can also be directly transmitted to human from bats or from human itself.

NiV was first discovered in Malaysia. From September 1998 to April 1999, a number of pig farm staffs died from severe encephalitis and a large number of pigs died of illness in Perak, Malaysia. It was initially thought to be a Japanese encephalitis virus infection, but it was later found that this outbreak was significantly different from Japanese encephalitis in terms of susceptible population, infection rate, and infection mode. In addition, many of the patients had been vaccinated against Japanese encephalitis, so the researchers identified this as a new infectious disease. In this outbreak, both people and livestock showed acute respiratory syndrome, resulting in 256 infections, 105 deaths, and 1.16 million pig deaths. The epidemic further spread to a slaughter house in Singapore, causing 2,511 workers infected, and 1 died. In October 1999, researchers isolated the virus from the cerebrospinal fluid of a patient. Shortly afterward, NiV was isolated from the urine of Malaysian fruit bat, the natural host of NiV was identified. Subsequently, Nipah virus disease has been reported in India, Cambodia, Thailand and other countries. In recent years, Nipah virus disease has occurred many times in Bangladesh and India, causing hundreds of deaths, with a mortality rate of 50% to 100%. Studies on NiV have mainly focused on the Malaysia strain (NiV-MY) and the Bangladesh strain (NiV-BD).

### Technical Problem

During the process of invading host cells, Nipah virus binds to the receptor ephrin-B2/B3 through the viral surface glycoprotein G, which will activate the conformation change of fusion glycoprotein (F), thereby mediate the fusion of the viral membrane and the cell membrane, and finally enable the viral genome to entry the cell. Both protein G and protein F are important targets in vaccine and antiviral drug development. There is currently no vaccine available for human use. As for drugs, only one monoclonal antibody, m102.4, has entered clinical trials. M102.4 is a human monoclonal antibody screened from recombinant human Fab phage display library. In addition to NiV, M102.4 can also effectively neutralize Hendravirus, another species belonging to the Henipavirus genus of the Paramyxoviridae family. In challenge protection experiments of ferrets and African green monkeys, m102.4 can achieve effective protection after Nipah virus challenge. In 2010, m102.4 was administered as an emergency protective drug in two individuals at high risk of exposure, neither of whom developed symptoms of infection.

In view of the technical demand for therapeutic antibodies against Nipah virus in the art, the purpose of the invention is to provide candidate monoclonal antibodies targeting unique epitopes on protein G, and then provide its application in the preparation of a medicine for the treatment of Nipah virus disease.

### Technical Solutions

Based on the above purpose, the invention firstly provides a specific neutralizing antibody against Nipah virus glycoprotein G. The said antibody is monoclonal antibody, and the amino acid sequence of CDR (complementarity determining region)1, CDR2, CDR3 of the heavy chain variable region of the said antibody and the amino acid sequence of CDR1, CDR2, CDR3 of the light chain variable region of the said antibody are respectively shown as the following sequence combinations:
26-33, 51-58, 97-110 of SEQ ID NO:1 and 26-34, 52-54, 91-100 of SEQ ID NO:3, or
26-33,51-58, 97-105 of SEQ ID NO:5 and 27-37, 55-57, 94-107 of SEQ ID NO:7, or
26-33, 51-58, 97-110 of SEQ ID NO:9 and 26-34, 52-58, 97-105 of SEQ ID NO:11, or
26-33, 51-58, 97-108 of SEQ ID NO:13 and 27-37, 55-57, 94-102 of SEQ ID NO:15.

In a preferred embodiment, the amino acid sequence of the heavy chain variable region of the said antibody and the amino acid sequence of the light chain variable region of the said antibody are respectively shown as any combination of the following sequences:
SEQ ID NO:1 and SEQ ID NO:3 (in the invention, the antibody with these variable regions is named as “1A9”), or
SEQ ID NO:5 and SEQ ID NO:7 (in the invention, the antibody with these variable regions is named as “1D11”), or
SEQ ID NO:9 and SEQ ID NO:11 (in the invention, the antibody with these variable regions is named as “1F9”), or
SEQ ID NO:13 and SEQ ID NO:15 (in the invention, the antibody with these variable regions is named as “2B6”).

In a more preferred embodiment, the amino acid sequence of the heavy chain constant region of the said antibody is shown as SEQ ID NO:17, and the amino acid sequence of the light chain constant region of the said antibody is shown as SEQ ID NO:19 or SEQ ID NO:21.

Secondly, the invention also provides an isolated nucleic acid encoding the heavy chain and/or light chain of the said monoclonal antibody. The sequence of the isolated nucleic acid encoding the heavy chain variable region and/or the sequence of the isolated nucleic acid encoding the light chain variable region are respectively shown as any combination of the following sequences:
SEQ ID NO:2 and SEQ ID NO:4 (in the invention, the antibody with these variable regions is named as “1A9”), or
SEQ ID NO:6 and SEQ ID NO:8 (in the invention, the antibody with these variable regions is named as “1D11”), or
SEQ ID NO:10 and SEQ ID NO:12 (in the invention, the antibody with these variable regions is named as “1F9”), or
SEQ ID NO:14 and SEQ ID NO:16 (in the invention, the antibody with these variable regions is named as “2B6”).

In a preferred embodiment, the isolated nucleic acid encoding the heavy chain constant region is shown as SEQ ID NO:18, and the isolated nucleic acid encoding the light chain constant region is shown as SEQ ID NO:20 or SEQ ID NO:22.

Thirdly, the invention also provides a functional element expressing the above-mentioned isolated nucleic acid encoding the heavy chain and/or light chain of the said monoclonal antibody.

In a preferred embodiment, the functional element is a linear expression cassette.

In another preferred embodiment, the functional element is a mammalian expression vector.

Fourthly, the invention also provides a host cell comprising the above functional elements.

In a preferred embodiment, the host cell is Expi293F cell.

In another preferred embodiment, the host cell is CHO-S cell. In the invention, CHO-S cell can be used to construct stable expression cell lines to realize industrial production.

Finally, the invention provides an application of the above-mentioned monoclonal antibodies in the preparation of the therapeutic drug for Nipah virus disease.

### Technical Effect

The monoclonal antibodies against Nipah virus glycoprotein G in the invention are composed of monkey-derived variable region and human-derived constant region, and the monkey-derived light and heavy chains variable region have unique CDR regions. Antibodies disclosed in the invention exhibit excellent capacity of binding with antigen, and can effectively bind with glycoprotein (G) of Malaysia and Bangladesh Nipah virus. The antibodies can potently neutralize the pseudotyped Nipah virus. The neutralizing capacity of the antibody increased with the increase of antibody concentration, and nearly 100% inhibition against Nipah pseudovirus could be achieved at a concentration of 1 μg/mL. The invention also discloses the application of the said monoclonal antibodies against Nipah virus glycoprotein G in the preparation of the therapeutic drug for Nipah virus disease.

### BRIEF DESCRIPTION OF THE FIGURES

FIG. 1. Sorting of rhesus monkey memory B cells;

FIG. 2. Capillary electrophoresis of nested PCR products;

FIG. 3. Distribution of ELISA 013450-630 nm values for screening of binding antibodies;

FIG. 4. The curves of antibody binding with antigen in ELISA detection;

FIG. 5. The neutralization capacity of mAbs against NiV-BD pseudovirus;

FIG. 6. The neutralization curves of mAbs against NiV-MY pseudovirus;

FIG. 7. The curves of mAbs competitively inhibiting to the binding of NiV-BD/MY G protein to receptor ephrin-B2/B3.

### EXAMPLES

The invention is further described below with reference to specific embodiments, and the advantages and characteristics of the invention will become clearer with the description. However, these embodiments are only exemplary, and do not constitute any limitation on the protection scope defined by the claims of the invention.

### Example 1. Screening and Preparation of Antibodies

### 1. Collection of Blood Samples

Female rhesus monkeys were immunized with adenovirus vector Nipah virus candidate vaccine, recombinant NiV G protein or recombinant HeV G protein three times by intramuscular injection on day 0, 28, and 49, respectively. Finally, blood samples of the rhesus monkey were collected on day 77.

2. Labeling of NiV-BD G with FITC

The NiV-BD G was labeled with fluorescein isothiocyanate (FITC) to sort antigen-specific memory B cells. Method is described as below:
1) FITC (SIGMA, F4274) is dissolved in dimethyl sulfoxide at a final concentration of 20 mg/mL. Take 100 μL of NiV-BD G (3.3 mg/mL) and add carbonate buffer (pH=9.6) to 400 μL.
2) Add 8 μL FITC to the NiV-BD G solution and incubate for 3 h at 4° C. in the dark.
3) Buffer-exchange the protein into PBS using a 30 kDa centrifugal concentrator tube until the filtrate is transparent and colorless. Wrap the labeled protein in tin foil paper and store it at 4° C. until use.

### 3. Flow Sorting of Memory B Cells

PBMCs are isolated from blood samples using a Ficoll density gradient centrifugation method, details are described as follows:
1) Take fresh EDTA anticoagulant whole blood and dilute the whole blood with the same volume of PBS.
2) Add the separation solution to the centrifuge tube, and slowly spread the diluted blood sample above the surface of the separation solution and keep the interface between the two liquid surfaces clear. Separation solution, anticoagulated blood, PBS (or normal saline) volume is 1:1:1.
3) After Balancing, the tube is centrifuged at 800×g, 3rd gear acceleration and deceleration, room temperature, for 30 min. After centrifugation, the bottom of the tube is red blood cells, the middle layer is the separation solution, the top layer is the plasma/tissue homogenate layer. The thin and dense white film between the plasma layer and the separation solution layer is mononuclear cells (including lymphocytes and monocytes). Carefully pipette the mononuclear cells into another centrifuge tube.
4) Dilute the cells with PBS and gently invert and mix well. The tube is centrifuged at 300×g, room temperature, for 10 min. Discard supernatant and repeat twice. Finally, the lymphocytes were resuspended in PBS for later use.
5) Count 5×10⁵ cells in a volume of 50 μL PBS, add the five fluorescent dyes recommended in the following table 1, and incubate for 1 h at 4° C. in the dark.

TABLE 1
Fluorescent dyes used for cell sorting

| Marker | Fluorescence | Company/Cat. No. | Volume | |
|---|---|---|---|---|
| Antigen | FITC | SIGMA, F4274 | 4 | μg |
| IgG | PE | BD, 555787 | 15 | μL |
| CD19 | APC-AF 700 | Beckman, IM2470 | 5 | μL |
| CD3 | PerCP | BD, 552851 | 10 | μL |
| CD27 | PC7 | Beckman, A54823 | 10 | μL |

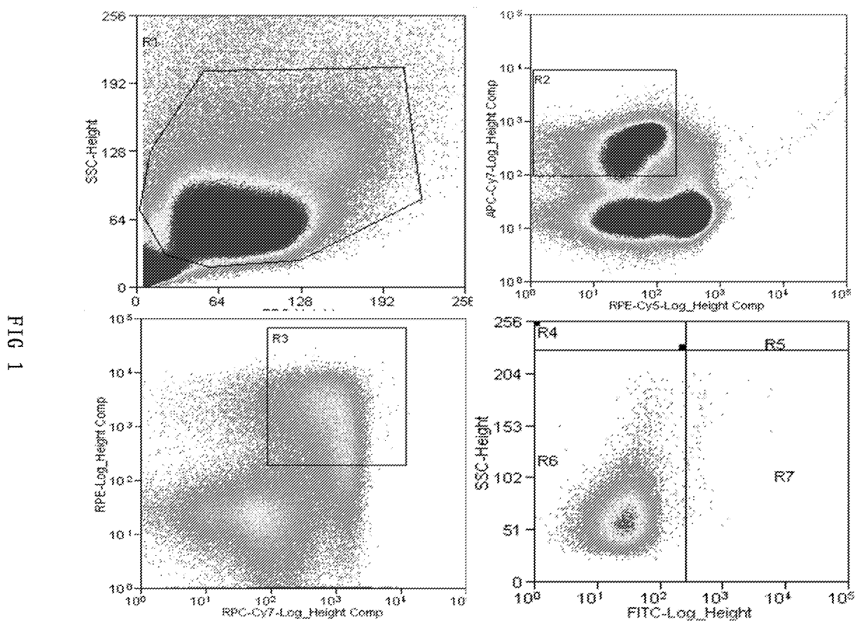

6) Wash cells 2-3 times with PBS containing 2% FBS and resuspend in 400 μL FPBS. Remove cell clusters with a 40 μm cell filter, and store at 4° C. in the dark for sorting.
7) NiV-BD G-specific single memory B cells are sorted by a cell sorter (Beckman, MoFlo XDP) using a strategy of IgG⁺/CD3⁻/CD19⁺/CD27⁺/NiV-BD G⁺. Each single cell is directly sorted into 96-well plates contains 20U RNase inhibitor and 204, RNase free water in each well. Store plates at −80° C. Cell sorting result is shown in FIG. 1. Cells circled by R7 box in the figure are characterized by IgG⁺/CD3⁻/CD19⁺/CD27⁺/NiV-BD G⁺, which are NiV-BD G-specific memory B cells.

### 4. Amplification of Antibody Genes by Single Cell PCR

1) Reverse Transcription PCR

A total of 1124 NiV-BDG-specific memory B cells were obtained by flow sorting. The SuperScript III reverse transcription kit was used to perform reverse transcription polymerase chain reaction (PCR). The mixed system was prepared according to the instructions and directly added to 96-well plates containing single cells for PCR reaction. The reaction system and conditions are described as follows.

Reaction conditions: 42° C., 10 min; 25° C., 10 min; 50° C., 60 min; 94° C., 5 min. The reaction system is described as table 2.

TABLE 2
Reaction system for reverse transcription PCR

| Component | Volume | |
|---|---|---|
| Template (sorted single cells) | 20 | μL |
| Random primer | 3 | μL |
| dNTP | 1 | μL |
| 10× buffer | 3 | μL |
| 0.1M DTT | 1 | μL |
| MgCl₂ | 2 | μL |
| RNaseOUT | 1 | μL |
| SuperScript III | 0.5 | μL |

2) Nested PCR

Reverse transcription products were used as the template, and two rounds of nested PCR reactions were performed to amplify H, κ, and λ genes. The detail process is described as follows.

The first-round nested PCR reaction system is listed in Table 3.

TABLE 3
The reaction system for the first-round of nested PCR

| Component | Volume | |
|---|---|---|
| Template (Reverse transcription products) | 1 | μL |
| Mixed primers (H/κ/λ) | 1.5/1/1 | μL |
| dNTP | 2 | μL |
| 10× buffer | 2.5 | μL |
| TransStart Taq DNA polymerase | 0.5 | μL |
| RNase-free water | to 25 | μL |

The first-round of nested PCR reaction conditions: firstly pre-denaturation 5 min at 95° C.; then 40 cycles of denaturation 30 s at 95° C., annealing 30 s at 57° C. and elongation 45 s at 72° C.; finally elongation 10 min at 72° C.

The first-round nested PCR primers are listed in Table 4.

TABLE 4
Primers for first-round nested PCR

| Primer | Sequence |
|---|---|
| 5′VH1.L1 | ATGGACTKGACCTGGAGG |
| 5′VH2.L1 | ATGGACACGCTTTGCTCC |
| 5′VH3A.L1 | ATGGAGTTKGGGCTGAGCTG |
| 5′VH3B.L1 | ATGGAGTTTGKRCTGAGCTGG |
| 5′VH3C.L1 | ATGGAGTCRTGGCTGAGCTGG |
| 5′VH3D.L1 | ATGGAGTTTGTGCTGAGTTTGG |
| 5′VH4.L1 | ATGAAGCACCTGTGGTTC |
| 5′VH5A.L1 | ATGGGGTCAACTGCCATC |
| 5′VH5B.L1 | ATGGGGTCCACCGTCACC |
| 5′VH6.L1 | ATGTCTGTCTCCTTCCTCA |
| 5′VH7.L1 | ATGGACCTCACCTGGAGC |
| 3′IgG(Outer) | GGAAGGTGTGCACGCCGCTGGTC |
| 5′VK1A.L1 | ATGGACATGAGGGTCCCCGC |
| 5′VK1B.L1 | GGCTCCTKCTGCTCTGGCTC |
| 5′VK2.L1 | ATGARGYTCCCTGCTCAG |
| 5′VK3.L1 | ATGGAARCCCCAGCWCAGC |
| 5′VK4.L1 | ATGGTGTCACAGACCCAAGTC |
| 5′VK5.L1 | ATGGCATCCCAGGTTCASC |
| 5′VK6A.L1 | ATGTTGTCTCCATCACAACTC |
| 5′VK6B.L1 | ATGGTGTCCCCATTGCAACTC |
| 5′VK7.L1 | ATGGGGTCCTGGGCTCC |
| 3′Kappa(Outer) | GTCCTGCTCTGTGACACTCTC |
| 5′VL1.L1 | ATGGCCTGGTYYCCTCTC |
| 5′VL2/7/10.L1 | ATGGCCTGGRCTCTGCTCC |
| 5′VL3A.L1 | ATGGCCTGGATTCCTCTC |
| 5′VL3B.L1 | ATGGCCTGGACCTTTCTC |
| 5′VL3C.L1 | ATGGCCTGGACCCCTCCC |
| 5′VL4A.L1 | ATGGCCTGGGTCTCCTTC |
| 5′VL4B.L1 | ATGGCCTGGACCCCACTC |
| 5′VL5/11.L1 | ATGGCCTGGACTCCTCTC |
| 5′VL6.L1 | ATGGCCTGGGCTCCACTCC |
| 5′VL8.L1 | ATGGCCTGGATGATGCTTC |
| 5′VL9.L1 | ATGGCCTGGGCTCCTCTG |
| 3′Lamda(Outer) | TGTTGCTCTGTTTGGAGGG |

The second-round nested PCR reaction system is listed in Table 5.

TABLE 5
The reaction system for the second-round of nested PCR

| Component | Volume | |
|---|---|---|
| Template (Products of the first-round nested PCR) | 1.6 | μL |
| Mixed primers (H/κ/λ) | 3.2/1.6/1.6 | μL |
| dNTP | 3.2 | μL |
| 10× buffer | 4 | μL |
| TransStart Taq DNA polymerase | 0.8 | μL |
| RNase-free water | to 40 | μL |

The second-round nested PCR primers are listed in Table 6.

TABLE 6
Primers for second-round nested PCR

| | Primer | Sequence |
|---|---|---|
| H | 5′VH1A.SE | TGGCAGCAGCTACAGGTGC |
| | 5′VH1B.SE | TGACAGCAGCTACAGGCGC |
| | 5′VH1C.SE | TGGCAGCAGCAACAGGCAC |
| | 5′VH2.SE | GTCCCGTCCTGGGTCTTGTC |
| | 5′VH3A.SE | GCTGTTTGGAGAGGTGTCCAGTGTG |
| | 5′VH3B.SE | GCCATATTAGAAGGTGTCCAGTGTG |
| | 5′VH3C.SE | GCTCTTTTGAAAGGTGTCCAGTGTG |
| | 5′VH3D.SE | GCTATTTTAAGAGGTGTCCAGTGTG |
| | 5′VH3E.SE | GCTATTTTAAAAGGTGTCCAGTGTG |
| | 5′VH4.SE | AGCTCCCAGATGGGTCYTGTCC |
| | 5′VH5.SE | GCTGTTCTCCARGGAGTCTGTG |
| | 5′VH6.SE | GGCCTCCCATGGGGTGTC |
| | 5′VH7A.SE | GCAGCAACAGGTGCCCACTC |
| | 5′VH7B.SE | GCAGCAACAGGCACCCACTC |
| | 3′IgG(Inner) | GTTCAGGGAAGTAGTCCTTGAC |
| κ | 5′VK1/2.SE | CTCCCAGGTGCCAGATGTGA |
| | 5′VK1B.SE | GGTCCCTGGRTCCAGTGGG |
| | 5′VK3A.SE | TGGCTCCCAGGTACCACYGGA |
| | 5′VK3B.SE | TGGATCCCGGATGCCGCCG |
| | 5′VK3C.SE | TGGCTTCCGGATACCACTGGA |
| | 5′VK4.SE | CTGGATCTCTGGTGTCTGTGG |
| | 5′VK5.SE | CCTTTGGATCTCTGMTGCCAGG |
| | 5′VK6.SE | TGGGTTCCAGTCTCCAAGGG |
| | 5′VK7.SE | TGTGCTCCAGGCTGCAATGG |
| | 3′Kappa(Inner) | ATTCAGCAGGCACACAACAGAG |
| λ | 5′VL1A.SE | CTGTGCAGGGTCCTGGGCC |
| | 5′VL1B.SE | CTGCACAGGGTCCYGGGCC |
| | 5′VL2.SE | TCACTCAGGGCACAGGATCC |
| | 5′VL3A.SE | CGCCCTCTGCACAGTCTCTGTGG |
| | 5′VL3B.SE | CACTCTCTGCACAGGTTCCGTGG |
| | 5′VL4A.SE | TTCATTTTCTCCACAGGTCTCTGTG |
| | 5′VL4B.SE | CTTCACTGCAGAGGTGTCTCTC |
| | 5′VL5.SE | CACTGCACAGGTTCCCTCTC |
| | 5′VL6.SE | CTGCACAGGGTCTTGGGCTG |
| | 5′VL8.SE | GCTTATGGCTCAGGAGTGGA |
| | 3′Lamda(Inner) | AGACACACTAGTGTGGCCTTG |

The reaction conditions of the second-round of nested PCR are the same as those of the first-round of nested PCR.

3) Capillary Electrophoresis

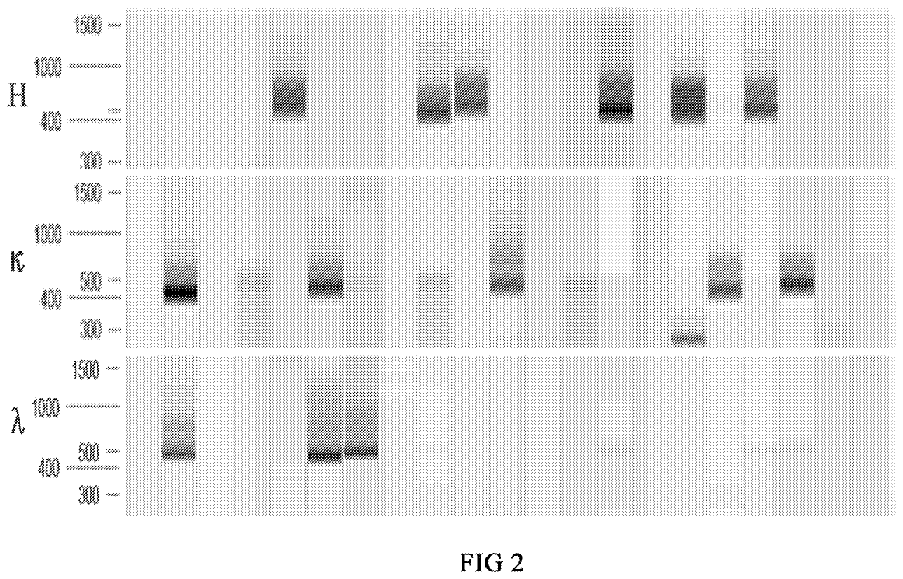

After the nested PCR, the amplified products were analyzed by capillary electrophoresis using the QIAxcel DNA Fast Analysis Cartridge. Positive clones with paired light and heavy chains were selected for sequencing, and the variable region sequences of the antibody were analyzed by Vector NTI software and IMGT website. The results of nested PCR capillary electrophoresis are shown in FIG. 2.

### 5. Expression of Antibodies Using Linear Expression Cassettes

Through the above single-cell PCR reaction, 254 paired antibody sequences were obtained, and the antibody was rapidly expressed by constructing linear expression cassettes.

Firstly, promoter-leader sequence fragments, constant region fragments (synthetized by Sangon Biotech, the heavy chain constant region sequence is shown as SEQ ID NO:17, the DNA coding sequence is shown as SEQ ID NO:18; the constant region sequence of the kappa light chain is shown as SEQ ID NO:19, the DNA coding sequence is shown as SEQ ID NO:20; the constant region sequence of the lambda light chain is shown as SEQ ID NO:21, and the DNA coding sequence is shown as SEQ ID NO:22), and poly A-tail fragments (Genbank accession number: X03896.1) were obtained by PCR. Then amplify the antibody variable region fragments, and the PCR reaction system is listed in Table 7.

TABLE 7
Reaction system for amplifying variable region fragments

| Component | Volume | |
|---|---|---|
| Template (Products of the second-round nested PCR) | 0.5 | μL |
| Mixed primers | 0.3 | μL |
| dNTP | 2 | μL |
| 10× buffer | 2 | μL |
| TransStart Taq DNA polymerase | 0.25 | μL |
| Deionized water | to 20 | μL |

PCR reaction conditions: firstly pre-denaturation at 95° C. for 5 min; then 30 cycles of 95° C., 30 s; 60° C., 30 s and 72° C., 30 s; finally elongation at 72° C. for 10 min.

Take the amplified promoter-leader sequence fragment, constant region-poly A tail fragment and variable region fragment as templates, and use CMV-UP and TK-PolyA as primers, to perform overlapped extension PCR to amplify linear expression cassettes of H, κ and λ chains. PCR products were identified by nucleic acid electrophoresis. The reaction system for amplifying the full-length linear expression cassettes is listed in Table 8.

TABLE 8
Reaction system for amplifying full-
length linear expression cassettes

| Component | Volume | |
|---|---|---|
| Template 1 (promoter-leader sequence fragment) | 10 | ng |
| Template 2 (constant region-poly A tail fragment) | 10 | ng |
| Template 3 (variable region fragment) | 0.5 | μL |
| Upstream primer (CMV-UP) | 2.5 μL (10 μM) | |
| Downstream primer (TK-Poly A) | 2.5 μL (10 μM) | |
| dNTP | 4 | μL |
| 10× buffer | 5 | μL |
| TransStart Taq DNA polymerase | 1 | μL |
| Deionized water | to 50 | μL |

PCR reaction conditions: pre-denaturation at 95° C. for 5 min; 95° C., 30 cycles of 30 s; 60° C., 30 s; 72° C. and 3 min; finally elongation at 72° C. for 10 min.

PCR reaction products were directly recovered with the OMEGA kit and quantified with Nano (GE Healthcare). One day before transfection, 2×10⁴ cells in 150 μL medium were seeded into each well of 96-well plates. On the day of transfection, took 0.2 μg of each light and heavy chain, added 0.8 μL of Turbofect transfection reagent, diluted to 40 μL with DMEM medium, and incubated at room temperature for 15 min after mixing. The mixtures were slowly added dropwise to 96-well plates and then cultured in a 37° C. incubator for 48 h.

6. Screening of Antibodies with Binding Capacity by ELISA
1) One day before the experiment, microplates are coated with 100 μL of NiV-BD G at 1 μg/mL and incubated overnight at 4° C. in a humid box.
2) On the following day, plates are washed 5 times with a plate washer (BIO-TEK, 405_LS). After adding 100 μL of blocking buffer to each well, plates are incubated at 37° C. for 1 h.
3) After washed 5 times, plates are added 100 μL of the transfected cell culture supernatant and then incubated at 37° C. for 1 h.
4) After washed, plates are added 100 μL of HRP-labelled goat anti-human IgG—(Abcam, Ab97225) at a dilution of 1:10,000, and then incubated at 37° C. for 1 h.
5) After washed, plates are added 100 μL of TMB substrate for 6 min in the dark at room temperature, followed by addition of 50 μL stop solution. Optical density at a 450-630 nm is read on a microplate reader.

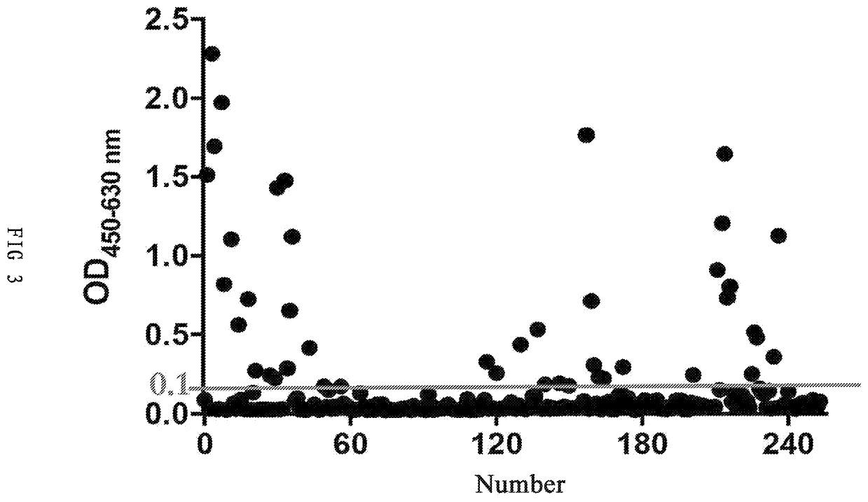

Results: Taking 0.1 as the cutoff value of optical density, Fifty-nine antibodies that can specifically bind to NiV-BDG were screened from 254 amplified positive clones. These antibodies were further expressed, purified and verified. Distribution of OD values for ELISA screening of antibodies with binding capacity is shown in FIG. 3.

### 7. Construction of the Expression Plasmid and Preparation of the Antibody

The expression plasmids are constructed and then the antibodies are prepared by expression. The method is described as follows:
1) Full-length genes of heavy chain and light chain linear expression cassettes are digested with EcoR I (NEB, R3101) and Not I (NEB, R3189), then ligated into pcDNA3.4 expression plasmids.
2) 15 μg of pcDNA3.4-H and 15 μg of pcDNA3.4-L are co-transfected into 30 mL Expi293 system (Life, A14524), and cells are cultured at 125 rpm, 5% CO₂ for 72 h.
3) The expression supernatant is collected by centrifugation at 3000×g for 10 min. Antibodies are purified using a rProtein A column. Antibodies are buffer-exchanged into PBS and then quantified by BCA protein quantification kit (Thermo Scientific, 23225).

### Example 2. Detection of the Binding Capacity of the Antibody by ELISA

1) One day before the assay, microplates are coated with 100 μL of NiV-BD G or NiV-MY G (NiV-BD G, Genbank: AY988601.1; NiV-MY G, Genbank: FN869553.1) at 1 μg/mL and incubated overnight at 4° C.
2) On the following day, after washing plates, adding 100 μL of blocking buffer is added into each well, plates are incubated at room temperature for 1 h.
3) Wash plates by plate-washer, then add 150 μL of purified antibodies at a concentration of 20 μg/mL to the first well, and add 100 μL of dilution solution to the remaining wells. Transfer 50 μL from the first well to the second well, and so on, dilute at a gradient of 1:3, with a final volume of 100 μL per well. Incubate plates for 1 h at room temperature.
4) Wash plates, add 100 μL of HRP-labelled goat anti-human IgG (Abcam, Ab97225) at a dilution of 1:10,000 into each well, and then incubated at room temperature for 1 h.
5) Wash plates, then add 100 μL of TMB substrate per well, and allow color reacting for 6 min in the dark at room temperature, followed by addition of 50 μL stop solution, finally read optical density at a 450-630 nm on a microplate reader.

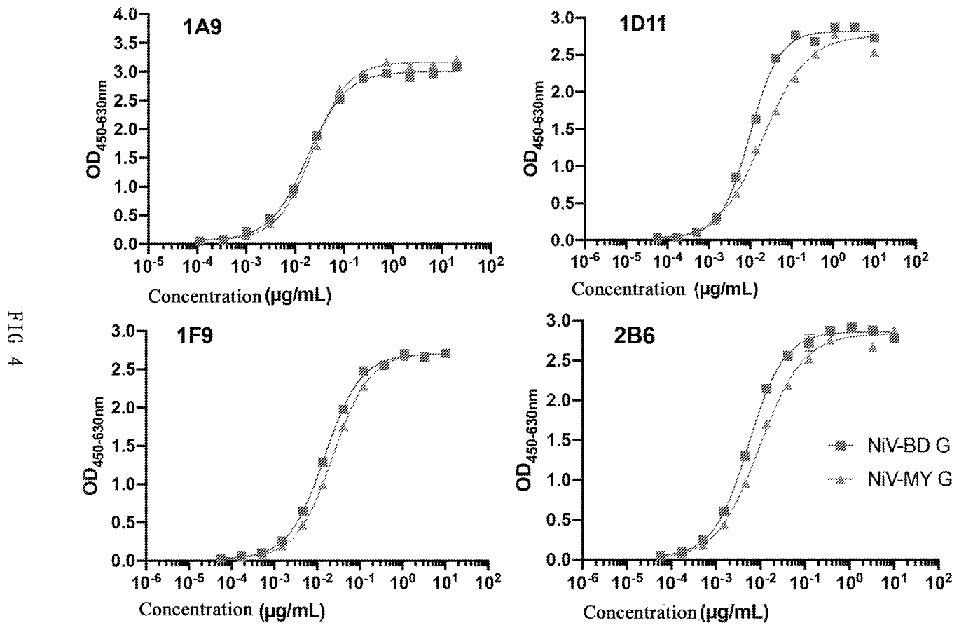

As shown in FIG. 4, antibodies have good binding capacity to the G protein of NiV-BD and NiV-MY. The half effective concentration (EC₅₀) values of antibody 1A9 are 18.26 ng/mL and 22.81 ng/mL, respectively; the EC₅₀ values of antibody 1D11 are 9.91 ng/mL and 20.2 ng/mL, respectively; the EC₅₀ values of antibody 1F9 are 15.04 ng/mL and 23.56 ng/mL, respectively; the EC₅₀ values of antibody 2B6 are 5.40 ng/mL and 9.56 ng/mL, respectively.

The above-mentioned four antibodies are sequenced. The nucleotide sequences of the heavy chain variable region of 1A9 is shown as SEQ ID NO:2; the nucleotide sequences of the light chain variable region of 1A9 is shown as SEQ ID NO:4; the amino acid sequences of the heavy chain variable regions of 1A9 is shown as SEQ ID NO:1; the amino acid sequences of the light chain variable regions of 1A9 is shown as SEQ ID NO:3; further analysis on the amino acid sequences of the heavy chain and the light chain variable region shows, the amino acid sequences of the CDR1, CDR2 and CDR3 region of the heavy chain variable region are respectively shown as 26-33, 51-58, and 97-110 of SEQ ID NO:1, the amino acid sequences of CDR1, CDR2 and CDR3 regions of the light chain variable region are respectively shown as 26-34, 52-54, and 91-100 of SEQ ID NO:3.

The nucleotide sequences of the heavy chain variable region of 1D11 is shown as SEQ ID NO:6; the nucleotide sequences of the light chain variable region of 1D11 is shown as SEQ ID NO:8; the amino acid sequences of the heavy chain variable regions of 1D11 is shown as SEQ ID NO:5; the amino acid sequences of the light chain variable regions of 1D11 is shown as SEQ ID NO:7; further analysis on the amino acid sequences of the heavy chain and the light chain variable region shows, the amino acid sequences of the CDR1, CDR2 and CDR3 region of the heavy chain variable region are respectively shown as 26-33, 51-58, and 97-105 of SEQ ID NO:5, the amino acid sequences of CDR1, CDR2 and CDR3 regions of the light chain variable region are respectively shown as 27-37, 55-57, and 94-107 of SEQ ID NO:7.

The nucleotide sequences of the heavy chain variable region of 1F9 is shown as SEQ ID NO:10; the nucleotide sequences of the light chain variable region of 1F9 is shown as SEQ ID NO:12; the amino acid sequences of the heavy chain variable regions of 1F9 is shown as SEQ ID NO:9; the amino acid sequences of the light chain variable regions of 1F9 is shown as SEQ ID NO:11; further analysis on the amino acid sequences of the heavy chain and the light chain variable region shows, the amino acid sequences of CDR1, CDR2 and CDR3 region of the heavy chain variable region are respectively shown as 26-33, 51-58, and 97-110 of SEQ ID NO:9, the amino acid sequences of CDR1, CDR2 and CDR3 regions of the light chain variable region are respectively shown as 26-34, 52-58, and 97-105 of SEQ ID NO:11.

The nucleotide sequences of the heavy chain variable region of 2B6 is shown as SEQ ID NO:14; the nucleotide sequences of the light chain variable region of 2B6 is shown as SEQ ID NO:16; the amino acid sequences of the heavy chain variable regions of 2B6 is shown as SEQ ID NO:13; the amino acid sequences of the light chain variable regions of 2B6 is shown as SEQ ID NO:15; further analysis on the heavy chain and light chain variable region amino acid sequences shows, the amino acid sequences of CDR1, CDR2 and CDR3 region of the heavy chain variable region are respectively shown as 26-33, 51-58, and 97-108 of SEQ ID NO:13, the amino acid sequences of CDR1, CDR2 and CDR3 regions of the light chain variable region are respectively shown as 27-37, 55-57, and 94-102 of SEQ ID NO:15.

The above four monoclonal antibodies have the same human heavy chain and light chain constant regions. The sequence of the polynucleotide encoding the heavy chain constant region is shown as SEQ ID NO:18, and the sequence of the polynucleotide encoding the light chain constant region is shown as SEQ ID NO:20 or SEQ ID NO:22, the amino acid sequence of the heavy chain constant region is shown as SEQ ID NO: 17, and the amino acid sequence of the light chain constant region is shown as SEQ ID NO:19 or SEQ ID NO:21.

### Example 3. Pseudovirus Neutralization Assay to Evaluate the Neutralizing Capacity of the Antibodies

Package of human immunodeficiency virus (HIV)-backbone NiV-BD and NiV-MY pseudoviruses to evaluate the neutralizing capacity of monoclonal antibodies in vitro (Dimple Khetawat, C. C. B., A functional henipavirus envelope glycoprotein pseudotyped lentivirus assay system. Virology Journal 2010. 7(312)). Method is as below:
1) Dilute antibodies with DMEM medium, add 75 μL of antibody at 5 μg/mL to the first well of 96-well culture plates, and add 50 μL of DMEM medium to the remaining wells.
2) Transfer 25 μL of liquid from the first well into the second well, mix well, and so on, dilute at a ratio of 1:3, and the final volume of each well is 50 μL. Add 50 μL pseudovirus to each well and incubate at 37° C. for 1 h.
3) Count 293T cells and seed 100 μL cells at a density of 2×10⁵ cells/mL to each well. Place the culture plate in a 37° C. incubator for 36-48 h.
4) Take out plates, then carefully remove the medium. Add 100 μL of cell lysate to each well and shake at 400 rpm for 15 min on a shaker. Centrifuge at 3000 rpm for 10 min at room temperature. After mixing the lyophilized detection substrate and buffer of the luciferase detection system (Promega, E1501), then fill them in the GLOMAX 96 Microplate Luminometer (Promega) detection loops. Transfer 20 μL of the lysis supernatant and read the fluorescence value. Calculate the protection rate of the antibody on the cells.

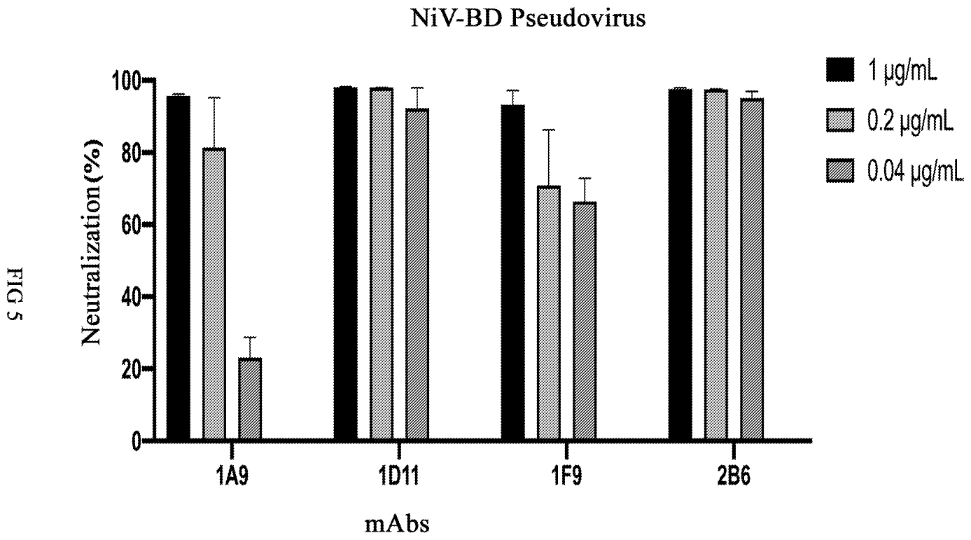

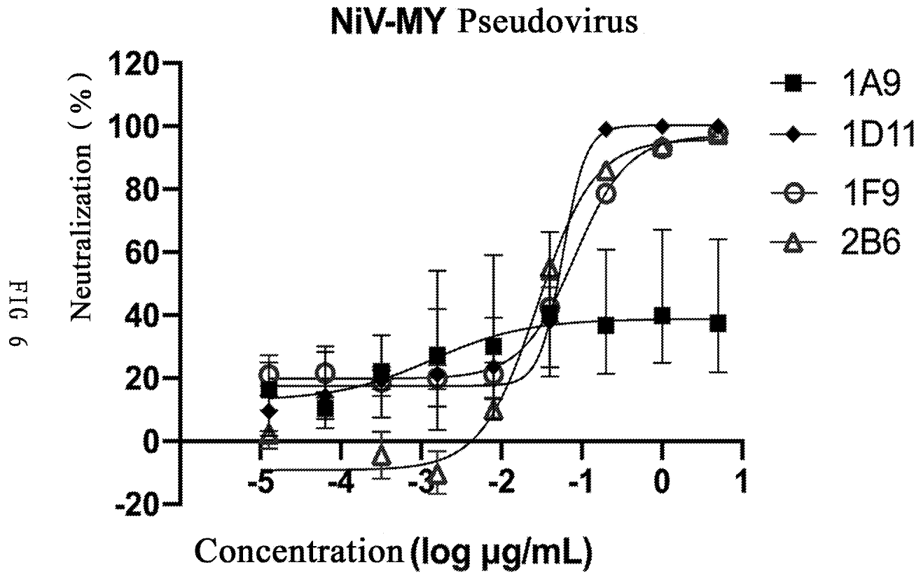

The results are shown in FIG. 5 and FIG. 6. The antibodies can effectively neutralize HIV-NiV-BD/MY pseudoviruses in vitro. The neutralizing capacity of the antibody increased with the increase of its concentration. As for 1D11, 1F9 and 2B6, nearly 100% neutralization against the two pseudotyped Nipah viruses could be achieved at a concentration of 1 μg/mL. However. 1A9 can only neutralize the NiV-BD pseudovirus, and has poor neutralization effect against the NiV-MY pseudovirus. For NiV-BD pseudovirus, monoclonal antibodies 1D11 and 2B6 have higher neutralization capacity than 1A9 and 1F9, and their neutralization rate can still reach over 95% at a concentration of 0.04 μg/mL; for NiV-MY pseudovirus, the half inhibiting concentration (IC₅₀) values of 1D11, 1F9 and 2B6 are 57.87 ng/mL, 81.41 ng/mL and 28.26 ng/mL, respectively.

### Example 4. Competition Experiment

The capacity of monoclonal antibody inhibiting the binding of Nipah virus G protein with the receptor was evaluated through Luminex microsphere competition inhibition assay. The method is described as follows:
1) Add 10 μL of 10 μmonoclonal antibody to the first well, and then dilute it by two times successively.
2) Add 1.25 ng of receptor ephrin-B2 or ephrin-B3 to each well in a volume of 10 μL. Add 10 μL of prepared microspheres (containing 1500 NiV-BD/MY G-coupled microspheres respectively) to each well, and incubate on a shaker for 60 min.
3) Add 10 μL of SAPE (concentration of 12 μg/mL) to each well and incubate on a shaker for 30 min.
4) Wash 3 times with 100 μL assay buffer and read by Luminex MAGPIX instrument.

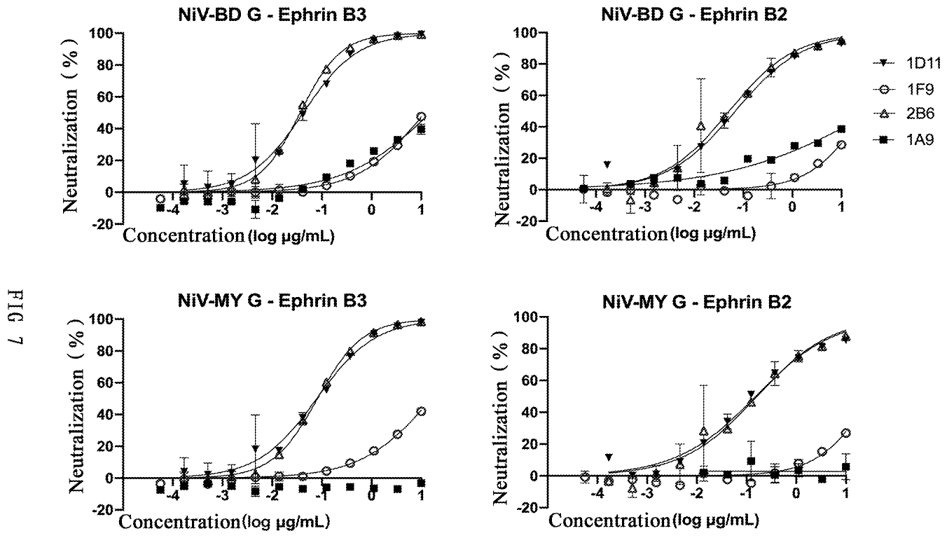

The curves of antibodies competitively inhibiting the binding of Nipah virus G protein with receptor ephrin-B2/B3 are shown in FIG. 7. The results show that 1D11 and 2B6 can effectively inhibit the binding of Nipah virus G protein with receptor ephrin-B2/B3 binding. The IC₅₀ values of 1D11 and 2B6 for inhibiting the binding of Nipah virus G protein with the receptor ephrin-B2/B3 ranged from 0.04 μg/mL to 0.16 μg/mL, suggesting that antibodies 1D11 and 2B6 are likely to play a neutralizing role by inhibiting the binding of Nipah virus G protein with receptor ephrin-B2/B3, while 1A9 and 1F9 may have other neutralizing mechanisms.

### INDUSTRIAL APPLICABILITY

The invention provides a series of anti-Nipah virus monoclonal antibodies with neutralizing activity and their application in the preparation of medicines. The monoclonal antibodies are easy to be industrially produced and have industrial practicability.

1. A monoclonal antibody against Nipah virus glycoprotein G, wherein the amino acid sequence of CDR1, CDR2, CDR3 of the heavy chain variable region of the said antibody and the amino acid sequence of CDR1, CDR2, CDR3 of the light chain variable region of the said antibody are shown respectively as the following sequence combinations:
26-33, 51-58, 97-110 of SEQ ID NO:1 and 26-34, 52-54, 91-100 of SEQ ID NO:3, or
26-33, 51-58, 97-105 of SEQ ID NO:5 and 27-37, 55-57, 94-107 of SEQ ID NO:7, or
26-33, 51-58, 97-110 of SEQ ID NO:9 and 26-34, 52-58, 97-105 of SEQ ID NO:11, or
26-33, 51-58, 97-108 of SEQ ID NO:13 and 27-37, 55-57, 94-102 of SEQ ID NO:15.

2. The monoclonal antibody of claim 1, wherein the amino acid sequence of the heavy chain variable region of the said antibody and the amino acid sequence of the light chain variable region of the said antibody are respectively shown as the following sequence combinations:
SEQ ID NO:1 and SEQ ID NO:3, or
SEQ ID NO:5 and SEQ ID NO:7, or
SEQ ID NO:9 and SEQ ID NO:11, or
SEQ ID NO:13 and SEQ ID NO:15.

3. The monoclonal antibody of claim 2, wherein the amino acid sequence of the heavy chain constant region of the said antibody is shown as SEQ ID NO:17, and the amino acid sequence of the light chain constant region of the said antibody is shown as SEQ ID NO:19 or SEQ ID NO:21.

4. An isolated nucleic acid encoding the heavy chain and/or the light chain of the monoclonal antibody of claim 1, wherein the sequence of the isolated nucleic acid encoding the heavy chain variable region and/or the sequence of the isolated nucleic acid encoding the light chain variable region are respectively shown as the following sequence combinations:
SEQ ID NO:2 and SEQ ID NO:4, or
SEQ ID NO:6 and SEQ ID NO:8, or
SEQ ID NO:10 and SEQ ID NO:12, or
SEQ ID NO:14 and SEQ ID NO:16.

5. The isolated nucleic acid of claim 4, wherein the sequence of the isolated nucleic acid encoding the heavy chain constant region is shown as SEQ ID NO:18, and the sequence of the isolated nucleic acid encoding the light chain constant region is shown as SEQ ID NO:20 or SEQ ID NO:22.

6. A functional element expressing the isolated nucleic acid of claim 5.

7. The functional element of claim 6, wherein the functional element is a linear expression cassette or a mammalian expression vector.

8. A host cell comprising the functional element of claim 7.

9. The host cell of claim 8, wherein the cell is Expi 293F cell or CHO-S cell.

10. Application of the monoclonal antibody of claim 1 in the preparation of a therapeutic drug for nipah virus disease.
